(19) United States
(12) Patent Application Publication
(10) Pub. No.: US 2006/0165641 A1
(43) Pub. Date: Jul. 27, 2006

(54) Cosmetic compositions containing combinations of hydroxamate derivatives and antioxidants in a liposomal delivery system

(76) Inventors: Kumar Pillai, Wayne, NJ (US); Brian Costello, Lakegrove, NY (US); Christian Oresajo, Nanuet, NY (US); Joseph Ceccoli, Farmingville, NY (US)

Correspondence Address: ENGELHARD CORPORATION, 101 WOOD AVENUE, ISELIN, NJ, 08830, US

(21) Appl. No.: 11/332,971

(22) Filed: Jan. 17, 2006

Related U.S. Application Data
(60) Provisional application No. 60/644,786, filed on Jan. 18, 2005.
(60) Provisional application No. 60/677,596, filed on May 4, 2005.

(51) Int. Cl. A61K 8/42

(52) U.S. Cl. 424/70.22; 424/401

(57) ABSTRACT

A cosmetic skin care composition includes salicylhydroxamate or one or more other hydroxamate derivatives in an amount of about 0.001% to about 10.0% by weight, and nordihydroguaiaretic acid or one or more other antioxidants in an amount of about 0.00001% to about 10% by weight. The composition can be optionally encapsulated in a liposomal vehicle.

This patent claims the benefit of pending U.S. Ser. No. 60/644,786 filed on Jan. 18, 2005 and 60/677,596 filed May 4, 2005 incorporated in their entirety herein by reference.

### TECHNICAL FIELD

The present invention is directed to combinations of hydroxamates and anti-oxidants, preferably encapsulated in a liposome, to deliver synergistic anti-inflammatory, anti-oxidant, anti-ageing and skin conditioning benefits in a cosmetic product.

### DISCUSSION OF THE RELATED ART

Antioxidants are compounds that can protect cells against the damaging effects of reactive oxygen species (ROS), such as singlet oxygen, superoxide, peroxyl radicals, hydroxyl radicals and peroxynitrite. An imbalance between antioxidants and reactive oxygen species can result in oxidative stress, leading to cellular damage. Oxidative stress has been linked to cancer, aging, atherosclerosis, ischemic injury, inflammation and neurodegenerative diseases (e.g. Parkinson's and Alzheimer's) and skin aging. One class of plant-derived antioxidants, flavonoids, have aroused interest recently because of their potential beneficial effects with regard to antiviral, anti-allergic, antiplatelet, anti-inflammatory, antitumor and antioxidant activities.

Topical application of antioxidants and flavonoids can protect skin from UV induced damage. Free radicals form in skin upon ultraviolet exposure. Acute and chronic photo radiation damage depletes the body's natural antioxidant enzyme systems and increases oxidative protein modifications, causing cross-links. These pathological effects are found in the upper and lower layers of the skin. Cross-linked or glycated proteins are classic characteristics of skin aging. Cross-linked proteins in the skin result in stiffening, wrinkling and the unsightly leathery appearance. Human studies have demonstrated pronounced protective effects of antioxidants when applied topically before ultraviolet radiation exposure. With respect to UVB-induced skin damage, the photoprotective effects of antioxidants are significant. Topical application of such combinations may result in a sustained antioxidant capacity of the skin, possibly due to antioxidant synergisms. Free radicals are a culprit behind UVA-induced skin alterations, thus indicating a basis for topical antioxidant administration. In a human study, topical application of antioxidants resulted in diminished severity of UVA-induced sun damage. Thus, regular application of skin care products containing antioxidants may be of the utmost benefit in efficiently preparing skin against exogenous oxidative stressors occurring during daily life.

Topical antioxidants can prevent generation of ROS and subsequent inflammatory reactions. ROS are generated either by the normal process of metabolism whereby excess electrons generated in the mitochondrial respiratory chain are donated to molecular oxygen to generate superoxide anions, or by UV-induced non-enzymatic conversion of molecular oxygen into superoxide anion radical. In addition, under certain circumstances UV and visible light are capable of causing excitation of molecular oxygen in the skin to form highly reactive singlet species.

Hydroxamates are a family of organic acids that are much weaker acids than the structurally related carboxylic acids. They are antibacterial, anti-fungal, and inhibitors of prostaglandin production, oxidoreductases such as perioxidases and tyrosinases, ureases, and matrix metalloproteases (MMPs). Hydroxamates also possess anti-inflammatory activities due to their ability to inhibit cyclooxygenase and lipooxygenase. Due to these properties, they are useful as ingredients in skin care products as anti-inflammatory, anti-aging, and skin lightening agents. Hydroxamates are also excellent chelators of metal ions in biological systems. Some examples of metal ion requiring enzymes that can be inhibited to provide skin benefits are matrix metalloproteases (Zn) for anti-aging benefits, tyrosinase (Cu) for skin lightening benefits. Several inhibitors of Zn containing MMPs have been developed based on the hydroxamation of small molecules. In other cases, hydroxamate complexes of metal ions can be used to provide skin benefits. These include: Ca for skin cell differentiation and ceramide synthesis; Cu and Zn for antioxidant activation, Sr and B for matrix stimulation, Mn, Mg, for integrin and extracellular matrix (ECM) component production, and Zn for increased turnover and metabolism of ECM.

Hydroxamates of salicylic acid are powerful inhibitors of cyclooxygenase and lipoxygenase, as disclosed in U.S. Pat. No. 6,696,477. These compounds have a variety of actions on plants, such as inhibition of alternate oxidase in plant and algal mitochondria, stimulation of seed germination and inhibition of redox enzymes. The mechanism of action of salicylhydroxamate need not necessarily be mediated by its ion chelating ability alone. The hydroxamic acid moiety can be bound to redox enzymes in the same manner as a substrate or by the formation of a charge transfer complex between hydroxamic acid and an electron-accepting group in the enzyme. All these activities provide an opportunity for salicylhydroxamate to be a potent skin care active.

The parent molecule of salicylhydroxamate, salicylic acid, is a mild acid that works as a keratolytic agent: it encourages the sloughing of dead skin cells. It is a safe, effective treatment for mild acne, oily skin, textural changes and post-inflammatory hyperpigmentation for patients of most skin types. Salicylic acid helps unclog pores to resolve and prevent acne lesions. It does not, however, have any effect on the production of sebum or the presence of P. acnes bacteria. In addition, salicylic acid has weak antifungal and anti-inflammatory activities, in particular the inhibition of arachidonic acid cascade in the production of inflammatory prostaglandins and leukotrienes.

Anti-oxidants, due to their ability to quench ROS, can prevent inflammation, UV-induced inflammation, and prevent skin ageing. Many of the inflammatory reactions induced by UV irradiation are initiated by ROS, which eventually activate the proinflammatory mediators such as prostaglandin, leukatriene and cytokine generation, causing further damage to cells and tissues. Anti-oxidants can block UV-induced reactive oxygen generation and further potentiate the anti-inflammatory and anti-aging activities of hydroxamates.

However, hydroxamates of amino acids or salicylic acid are very hydrophilic compounds. Most of these studies have been carried out in in-vitro systems (fibroblasts, melanocytes or keratinocytes in culture) where penetration and availability are not issues. In order for these molecules and their metal complexes to have any in vivo benefits, one needs to deliver these active compounds into the deeper, metabolically active layers of the skin. In particular, the stratum corneum will act as a barrier for penetration of these hydrophilic compounds into the skin. A delivery vehicle in the form of phospholipid nanoparticles, i.e. liposomes, can enhance their delivery through stratum corneum into the viable epidermal and the dermal layers of the skin, thus enabling these molecules to interact with the cellular enzymatic systems.

### SUMMARY OF THE INVENTION

According to one embodiment of the present invention, there is provided a combination of one or more hydroxamate derivatives and one or more antioxidants to deliver synergistic anti-aging and anti-inflammatory benefits to skin care products. According to another embodiment of the present invention, there is provided a combination of salicylhydroxamate and NDGA that can deliver synergistic anti-aging and anti-inflammatory benefits to skin care products. According to another embodiment of the present invention, these ingredients can be encapsulated in a stable liposome to protect them from degradation and to deliver them into the deeper layers of skin to provide improved cosmetic benefits. According to further embodiment of the present invention, a cationic liposome vehicle can deliver these actives to hair follicles and sebaceous glands for benefits in hair growth, sebum suppression and to prevent acne formation.

### BRIEF DESCRIPTION OF THE DRAWINGS

FIG. 1 is a list of natural polyphenolic antioxidants.

FIG. 2 is a table summarizing antioxidant activity of NDGA.

FIG. 3 is a bar chart showing inhibition by NDGA of UV-B induced TNF-a protein production by Keratinocytes.

FIG. 4 is a table showing the effect of NDGA on TNF-α induced MMP-9 expression.

FIG. 5 is a bar chart showing singlet oxygen scavenging activity of NDGA in comparison with Coenzyme Q10 and green tea polyphenols.

FIG. 6 is a table showing hydroxyl radical scavenging activity of NDGA in comparison with green tea polyphenols and Vitamin E.

FIG. 7 is a bar chart showing the anti-inflammatory effects of salicylic acid hydroxamate.

FIG. 8 is a table showing MMP-9 Inhibitory activity of Salicyl hydroxamate and Tryptophan hydroxamate.

FIG. 9 is a table showing induction of differentiation of keratinocytes by SHA.

FIG. 10 is a table showing fibroblast collagen synthesis stimulation by SHA.

FIG. 11 is a table showing SHA induced integrin expression of keratinocytes.

FIG. 12 is a table showing SHA induced integrin expression of fibroblasts.

FIG. 13 illustrates the structure of phospholipids and liposomes.

FIG. 14 depicts a phospholipid bilayer sphere according to an embodiment of the invention.

FIG. 15 is a bar chart showing the inhibition of tyrosinase by SHA.

FIG. 16 is a bar chart showing the anti-inflammatory activity of SHA.

FIG. 17 is a bar chart showing the inhibition of myeloperoxidase by SHA.

FIG. 18 is a bar chart showing the matrix metalloproteinase inhibition by SHA.

FIG. 19 is a bar chart showing the induction of terminal differentiation of keratinocytes by SHA.

FIG. 20 is a bar chart showing the induction of collagen synthesis by fibroblasts.

### DETAILED DESCRIPTION OF THE PREFERRED EMBODIMENTS

Exemplary embodiments of the invention as described herein generally include combinations of antioxidants and hydroxamates that can prevent the initiation of inflammation and inhibit the expression of pro-inflammatory mediators delivering a synergistic benefit to skin. In the interest of clarity, not all features of an actual implementation which are well known to those of skill in the art are described in detail herein.

The hydroxamates derivatives of compounds including, but not limited to, salicylic acid and its derivatives, hydroxybenzoic acid and other heterocyclic compounds, tryptophan, amino acids and small peptides (up to 6 amino acids in length), pseudo peptides, alpha hydroxy acids and their derivatives, and dicarboxylic acids, are useful. Metal ion complexes of hydroxamates can be formed from metal ions including Zn, Cu, Mn, Mg, Sr, B, Al, Rb, and Ca. Other useful hydroxamates include hydroxamate derivatives of substrate-analogue peptides of matrix metalloproteases containing aminomalonic acid, and monohydroxamates of aspartic and glutamic acids.

Useful anti-oxidants include, but are not limited to, vitamin E (tocopherol) and its esters, vitamin C and its derivatives, green tea polyphenols, coenzyme Q10, quercetin and other bioflavinoids, plant extracts such as grape seed, pomegranate, amla (emblica Officinale), and other polyphenolic antioxidants such as nordihydroguaiaretic acid (NDGA).

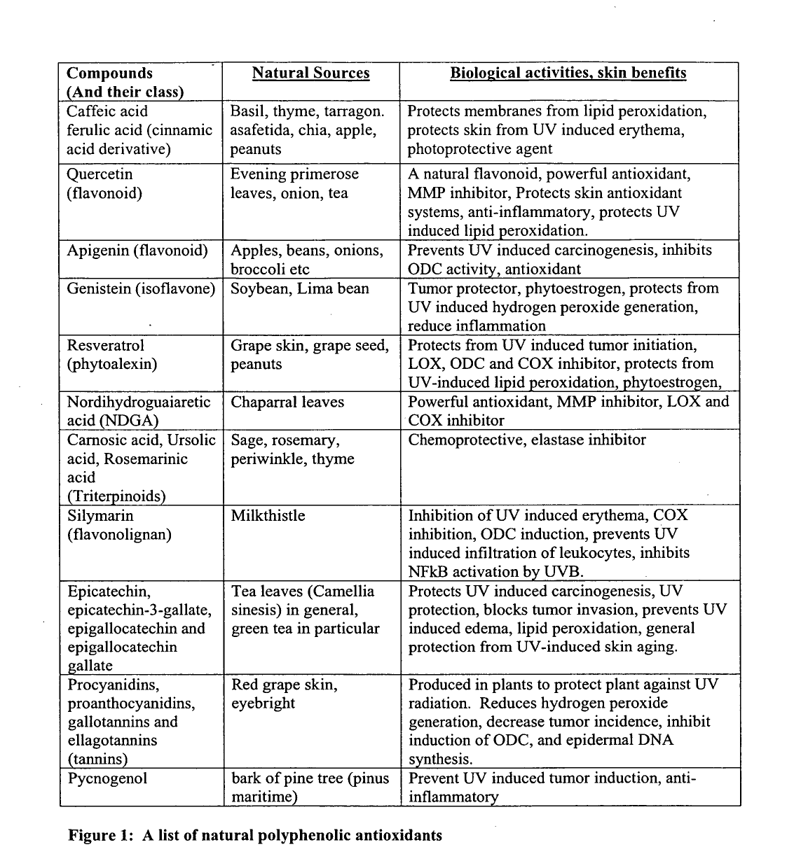

Most commonly used antioxidants useful for skin care include, but are not limited to, vitamins such as vitamin C and its derivatives, vitamin E (tocopherol) and its esters, ubiquinone, coenzyme Q10 and lipoic acid. In addition a variety of plant derived compounds have excellent antioxidant activities. These include flavonoids and polyphenols. Flavonoids are polyphenolic compounds that are ubiquitous in nature and are categorized, according to chemical structure, into flavonols, flavones, flavanones, isoflavones, catechins, anthocyanidins and chalcones. Over 4,000 flavonoids have been identified, many of which occur in fruits, vegetables and beverages such as tea, coffee, beer, wine and fruit drinks. The table in FIG. 1 summarizes some of the naturally occurring polyphenolic antioxidants useful in this application in combination with hydroxamates. The left column of the table lists exemplary polyphenolic antioxidants, the middle column list their respective natural sources, while the right column lists the biological activities and skin benefits of the antioxidants.

A cosmetic skin care composition according to one embodiment of the invention includes an antioxidant, such as NDGA, in an amount of from about 0.00001 to about 10 wt. %, and a hydroxamate derivative in an amount of about 0.001 to about 10 wt. %, encapsulated in a liposomal vehicle. The use of NDGA is illustrative, and other antioxidants are within the scope of the invention. The hydroxamate derivative can be one or more of salicylhydroxamate, aminoacid hydroxamate, or a hydroxamate derivative of a peptide. This list of hydroxamates is illustrative, and other hydroxamate derivatives are within the scope of the invention. In general, the amount of antioxidant in the inventive compositions is in the range of from about 0.00001 to about 10% by weight composition. Preferably, in order to lower cost and maximize the effect, the amount of antioxidant is in the range of from about 0.001% to about 5%, and most preferably is in the range of from about 0.01% to about 5%. The hydroxamate is generally employed in the inventive compositions in an amount of from about 0.001 to about 10%, preferably from about 0.01 to about 1%, most preferably from about 0.01 to about 0.5% by weight of the compositions.

According to another embodiment of the invention, a cosmetic composition comprises a combination of an antioxidant and a hydroxamate derivative in a liposome, including: (1) about 0.1% to about 10% by weight of a membrane-forming lipid phase; (2) about 0.00001% to about 10% by weight of antioxidant, preferably NDGA; (3) about 0.001% to about 10% by weight of hydroxamate derivative, preferably salicylhydroxamate; and (4) about 40% to about 99.8% by weight of water. The composition is subjected to high sheer and high pressure processing to form the liposome.

All weight percentages are based upon the total % of composition weight. It should be noted that one or more antioxidants can be combined with one or more hydroxamate derivatives. A single antioxidant and hydroxamate or a combination of different antioxidants and hydroxamates, in differing amounts, can be incorporated in the liposomes. The antioxidants and the hydroxamate derivatives may be selected from the lists disclosed herein. In addition, the use of liposomes as a delivery agent is optional, as skin washes, creams, or lotions, as are known in the art, can be formulated to incorporate an antioxidant-hydroxamate combination according to an embodiment of the invention.

NDGA as an Anti-Oxidant

Nordihydroguaiaretic acid (NDGA) is a phenolic compound that is a component of resinous exudates of many plants. It is an extremely potent antioxidant and is used for protection against oxidation of fats and oils in the food industry. Many of its biological activities are mediated by this antioxidant potential. Traditionally extracted from a plant (larrea divaricata), NDGA has been used for years as an oil-soluble antioxidant. When combined with other antioxidants such as tocopherol, ascorbic acid or citric acid, a powerful synergy of antioxidant protection is created. More recently, studies have revealed NDGA's biological properties as a free-radical scavenger, a protective agent against keratosis caused by exposure to UV light, and as a lipooxigenase inhibitor with potential applications to the treatment of psoriasis and other skin disorders.

NDGA is also a potent anti-inflammatory compound. It inhibits the synthesis of inflammatory mediators such as prostaglandins and leukotrines. NDGA prevents leukocyte infiltration into tissues and release of ROS. NDGA can also block chemically-induced skin irritation.

NDGA can block UV effects. It can reduce the damage caused by UVB on skin by blocking UV-induced gene activation of inflammatory cytokines in skin. UVB induced activation of MMP enzymes such as tumor necrosis factor-alpha (TNF-α) is mediated by up regulation of the activator protein-1 (AP-1) transcription factor. Retinoic acid prevents this activation. However, NDGA at very low levels inhibits AP-1 activation in keratinocyte cell line HaCaT cells. In addition, NDGA blocks matrix degradation. UVB induces the activity and expression of MMPs. NDGA blocks this effect by interfering with the UV effects on MMP activation. In particular, NDGA inhibits TNF-α induced MMP 13 mRNA and protein induction in both primary human chondrocytes and human chondrosarcoma cell line SW1353.

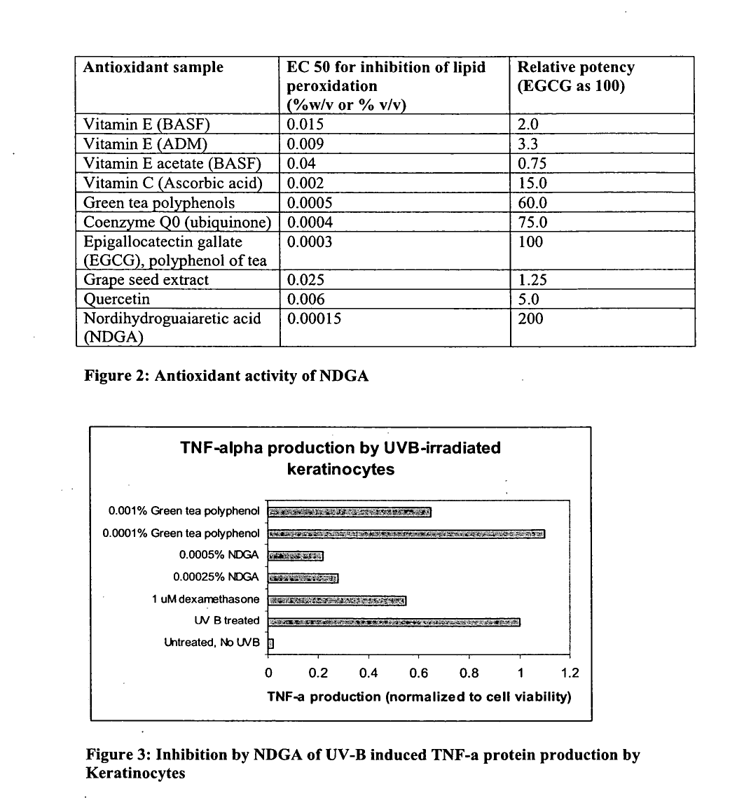

The ability of NDGA to prevent UV-induced lipid peroxidation was measured in comparison to other antioxidant industry standards such as vitamin E, vitamin C and green tea polyphenols. The assay was performed by irradiating a suspension of lecithin liposomes with UV light for 4 hours, in the absence or in the presence of the different compounds. Malonaldehyde levels (a breakdown product of oxidized lipids) were measured in aliquots of the reaction mixture at different times. The rate of lipid oxidation was calculated from the increase in malonaldehyde content. Relative potency was calculated by taking epigallocatechin gallate (EGCG) as 100%. These results are displayed in the table presented in FIG. 2. The antioxidant used in the comparisons are listed in the left column. The middle column lists the effective concentration at which the compound is 50% effective (EC 50) for inhibition of lipid peroxidation, while the right column lists the relative potency of the samples, as compared to polyphenol of tea, which is taken as 100. As can be seen from the table, NDGA is effective at a much lower EC 50 (0.00015) than the other samples, and is twice as potent as the next most effective antioxidant (EGCG), and is two orders of magnitude more potent than the vitamin E varieties.

The UV blocking effect of NDGA was measured by pretreating normal human keratinocytes with different concentrations of NDGA or other test compounds, then irradiating UVB with a single sub-lethal dose of 35 mJ/cm2 using a Phillips TL20W12 tube. This UVB treatment was used in FIG. 3 described below. Cells were further incubated for 24 hrs and TNF-α levels in the supernatant media samples were analyzed by enzyme linked immuno-sorbant assay (ELISA) kits. Dexamethasone was included as a positive control as an anti-inflammatory agent. Cell viability was also tested at the end of the 24 hr incubation period via metabolism of yellow tetrazolium salt of 3-(4,5-dimethylthiazolyl-2)-2,5diphenyltetrazolium bromide (MTT metabolism method). The data, presented in the bar graph of FIG. 3, was expressed as TNF alpha production normalized to cell viability. The top two bars indicate the production resulting from different concentrations of green tea polyphenols, the 3^{rd} and 4^{th} bars indicate the production resulting from different concentrations of NDGA, the 5^{th} bar the production due to dexamethasone, the 6^{th} bar the production from an untreated UVB irradiated cells, and the 7^{th} bar is the control (untreated cells, not UVB irradiated). The graph indicates that NDGA at low concentrations effectively blocked UVB-induced inflammation in keratinocytes. In addition, the graph indicates that NDGA was much more effective than dexamethasone or green tea polyphenols, a common antioxidant and anti-inflammatory ingredient used in cosmetic products.

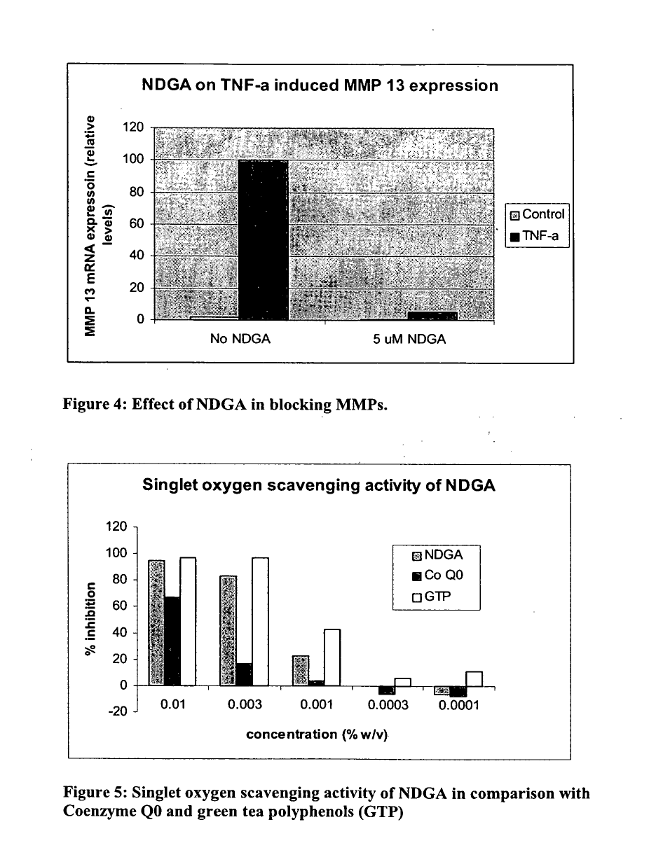

NDGA matrix metalloproteinase inhibition activity was measured by testing commercially available matrix metalloproteinase inhibitors (MMPi) for their ability to inhibit MMPs using a fluorogenic peptide assay system. In this system, the substrate is a fluorogenic peptide which, when cleaved by the MMP, undergoes an increase in fluorescence. The reaction is followed by monitoring the increase in the fluorescence of the reaction mixture for 2 hours. The rate of fluorescence increase is a measure of the reaction rate. The assay is performed in microtiter plates using triplicate wells per condition. The test sample contains specific MMP and the putative MMP inhibitor at several concentrations and the mean rate of the reaction at each of these conditions is compared to the reaction rate in the absence of inhibitor. The concentration of the test sample that inhibits the reaction rate (i.e., MMP inhibition) by 50% is defined as the “50% Inhibitory Concentration” or IC₅₀. This is the most commonly employed parameter indicating enzyme inhibition. A potent inhibitor has a low IC₅₀ value. The table in FIG. 4 shows the effect of NDGA on TNF-α induced MMP-9 expression as compared with different commercial samples, listed in the left column. The IC 50 values for MMP-9 (also known as gelatinase) inhibition are indicated in the middle column, while the relative potencies of the tested compounds are listed in the right column. As shown in the table, NDGA showed very high MMP-9 inhibitory activity. NDGA was several orders of magnitude more potent at much lower concentrations. IC₅₀ values were approximately 0.007% w/v or at 100 uM levels. This suggests that NDGA can be a potent inhibitor for MMP-9 inhibition.

Singlet forms of molecular oxygen are excited electronic configurations that are more reactive than the spin-restricted ground state. These forms can be generated in tissues by a variety of mechanisms. Particularly relevant to the skin is light-induced production of singlet oxygen. The singlet oxygen scavenging test is performed by irradiation of a reaction mixture that contains a photosensitizer dye (Rose Bengal) and iodide ions. The excited dye transfers its energy to ground state oxygen to generate singlet oxygen which in turn oxidizes iodide to I₃⁻. Production of I₃⁻ is followed spectrophotometrically at 355 nm. If a test sample is a singlet oxygen quencher, it will decrease the rate of I₃⁻ production. FIG. 5 is a bar graph that displays singlet oxygen scavenging activity of NDGA in comparison with Coenzyme Q10 (Coen Q10) and green tea polyphenols (GTP). The data is expressed as % inhibition of a control sample that does not contain any singlet oxygen quencher. The data indicate that NDGA is a very powerful singlet oxygen scavenger, equivalent to green tea polyphenols and better than coenzyme Q10.

The hydroxyl radical (—OH) is an extremely aggressive ROS that reacts with and damages all classes of biological molecules. It can be generated in tissues in a variety of ways. In areas of inflammation, where there are elevated levels of superoxide and free metal ions, .OH is generated from H₂O₂ via the well-known Fenton reaction. Hypochlorous acid (HOCl) also is present at elevated levels in inflamed tissues and its reaction with superoxide will generate additional .OH. Production of .OH in the skin is especially likely to occur as a result of homolytic fission of H₂O₂ initiated by exposure to UV light.

Hydroxyl radical scavenging of NDGA was measured by generation of .OH via the Fenton reaction with terephthalic acid as oxidizable substrate. Hydroxylation of terephthalic acid results in production of a fluorescent product. The rate of the reaction is determined by measuring the time course of fluorescence increase. If a test sample is a hydroxyl radical scavenger, it will decrease the reaction rate.

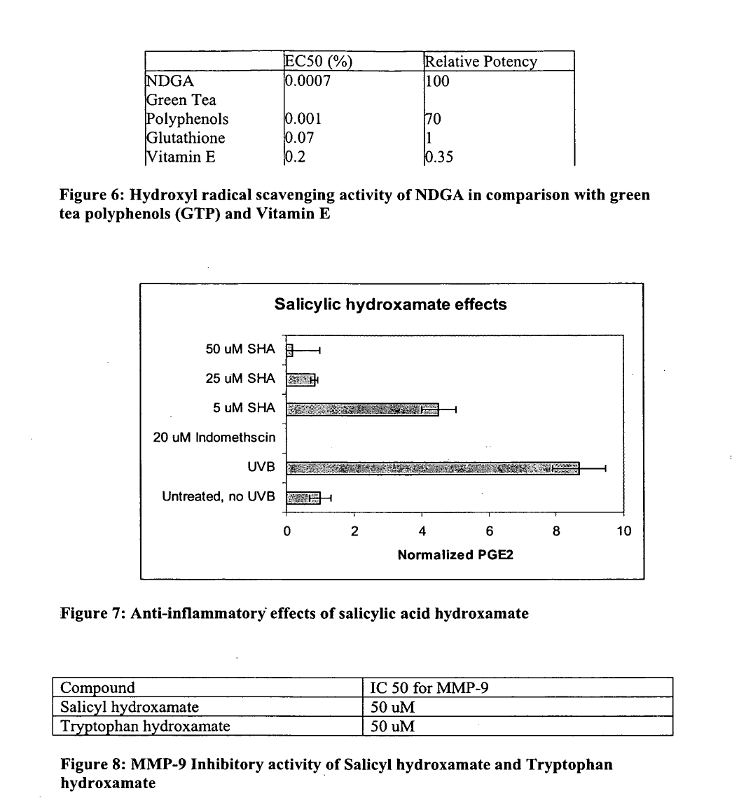

FIG. 6 is a table displaying hydroxyl radical scavenging activity of NDGA in comparison with green tea polyphenols (GTP) and Vitamin E, indicated in the left column. The data presented in the middle column shows the effective concentration at which the compound is 50% effective (EC₅₀ values) for inhibition of this hydroxyl radical-mediated aromatic hydroxylation, and the data presented in the right column shows the relative potency of the tested compounds. As can be seen from the data, NDGA is slightly more effective than green tea polyphenols and far more potent than glutathione or vitamin E as a hydroxyl radical scavenger.

Anti-Inflammatory Effects of Salicylhydroxamate (SHA)

The anti-inflammatory effect of SHA was measured by pretreating normal human keratinocytes with different concentrations of SHA for 24 hrs. The cells were UVB irradiated with a single sub-lethal dose of 30 mJ/cm2 using a Phillips TL20W12 tube. Cells were further incubated for 24 hrs with SHA and prostaglandin E2 (PGE2) levels in the supernatant media samples were analyzed by ELISA. 20 uM indomethacin was included as a positive control as an anti-inflammatory agent. The cell viability was also tested at the end of the 24 hr incubation period using the MTT metabolism method. FIG. 7 is a bar graph that displays the anti-inflammatory effects of the salicylic acid hydroxamate. The data was expressed as PGE2 production normalized to cell viability. PGE2, or prostaglandin E2, is an inflammatory mediator produced during inflammatory reactions in the skin. Any agents that inhibit the production of PGE2 will therefore have a beneficial effect of preventing inflammation (in other words, an anti-inflammatory effect). The top three bars display the effect of SHA at various concentrations on the UVB irradiated cells, the 4^{th} bar the effect of indomethscin, the 5^{th} bar the effect of UVB radiation on untreated cells, while the bottom bar is a control (untreated cells, not UVB irradiated). The data indicates potent anti-inflammatory effects of SHA at as low as 5 uM.

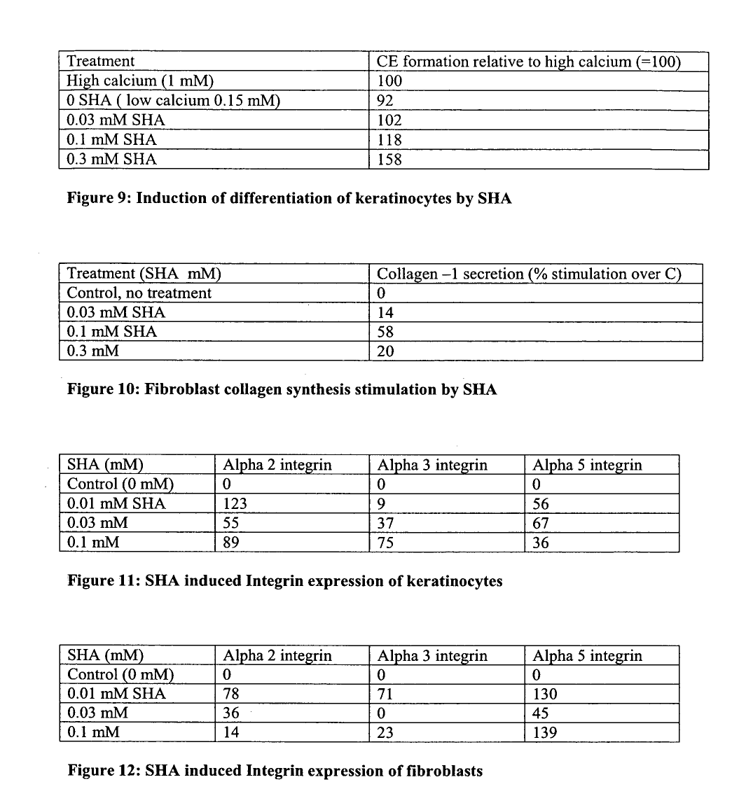

The MMP-9 inhibitory activity of salicyl hydroxamate and tryptophan hydroxamate were measured using the same experimental protocol as that used for measuring the effect of NDGA. FIG. 8 is a table that displays the inhibitory activity of Salicyl hydroxamate and Tryptophan hydroxamate, and indicates the IC 50 for MMP-9 in the right column. The data suggest that both salicyl hydroxamate and the hydroxamate derivative of an amino acid tryptophan are excellent and potent inhibitors of matrix metalloproteases.

SHA can induce cornified envelope formation of keratinocytes. This indicates the terminal differentiation of the epidermal cells. Cornified envelopes (CE) form the stratum corneum, the top protective layer of skin. Agents that induce its formation provide the skin with a protective barrier and improve skin condition.

To measure the induction of differentiation of keratinocytes, cultures of normal human keratinocytes were incubated with medium containing 0.15 mM calcium (low calcium prevents CE formation and keeps cells in growth mode). The cells were treated with different amounts of SHA and the amount of the highly cross-linked, detergent insoluble CE formation was quantified after dissolving the cells in 2% SDS/20 mM Dithiothrietol (DTT). The insoluble CE was centrifuged, resuspended in water and the optical density of the turbid solution was measured at 400 nm. The increase in turbidity indicates increased CE formation. FIG. 9 is a table that displays the induction of differentiation of keratinocytes by SHA at various concentrations. The results, presented in the right column, are expressed relative to high calcium (1 mM) containing cultures. These results indicate that SHA induces terminal differentiation of keratinocytes, increases the formation of CE, and thus improves skin condition.

In addition, SHA stimulates collagen and integrin synthesis of keratinocytes and fibroblasts. This was measured by treating keratinocyte or fibroblast cultures with SHA for 48 hrs and testing the medium for collagen and integrin secretion using appropriate antibodies using commercially available ELISA kits. The ELISA measures the secretion of collagen-1 and three different types of integrins, integrin alpha-2, -3 and -5. FIG. 10 is a table that displays fibroblast collagen synthesis stimulation by SHA at various concentrations. The data, presented in the right column, are expressed as a % stimulation of collagen secretion as compared to the control. The data indicate that 0.1 mM SHA significantly stimulated the collagen synthesis of fibroblasts. Stimulation of collagen synthesis would provide more integrity to the dermis and would benefit in reducing wrinkles and will provide anti-aging benefits.

SHA can also stimulate integrin expression of fibroblasts and keratinocytes. FIG. 11 is a table that displays SHA induced integrin expression of keratinocytes, while FIG. 12 is a table that displays SHA induced integrin expression of fibroblasts. In each table, that left column lists the various concentrations of SHA used in the tests, while the three right columns present integrin expression data, for different varieties of integrin, expressed as a % stimulation with respect to the control (0 mM SHA). The data presented indicate the effectiveness of SHA in increasing the integrin expression of keratinocytes and fibroblasts. Integrin is a vital component of the cell extracellular matrix that helps in cell adhesion and attachment. The increased amount of integrin helps cells to attach and grow, making skin firmer and more cellular. These attributes allow the skin to be plumper and moister, as well as reducing wrinkles and improving skin condition
Skin Lightening Effects of SHA

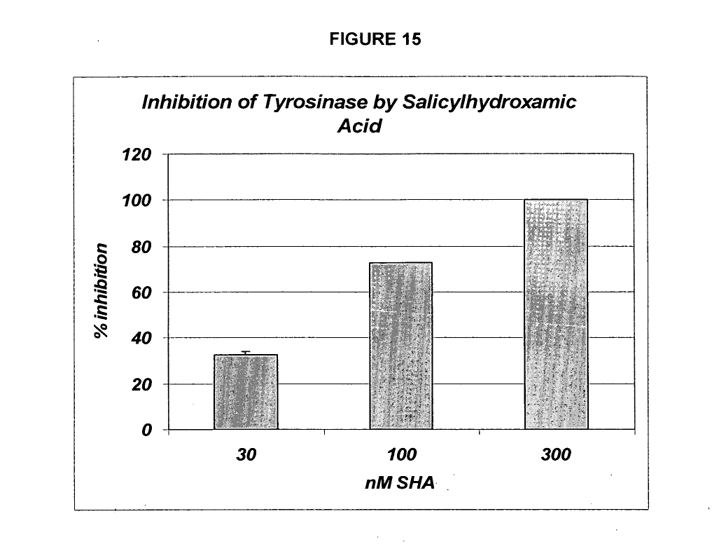

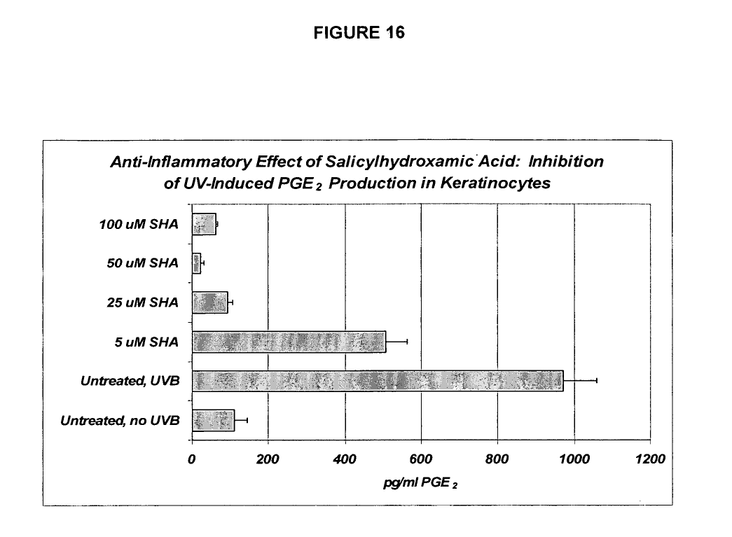

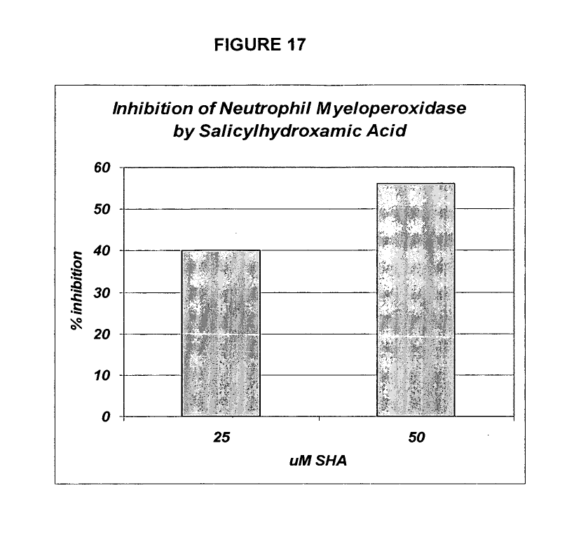

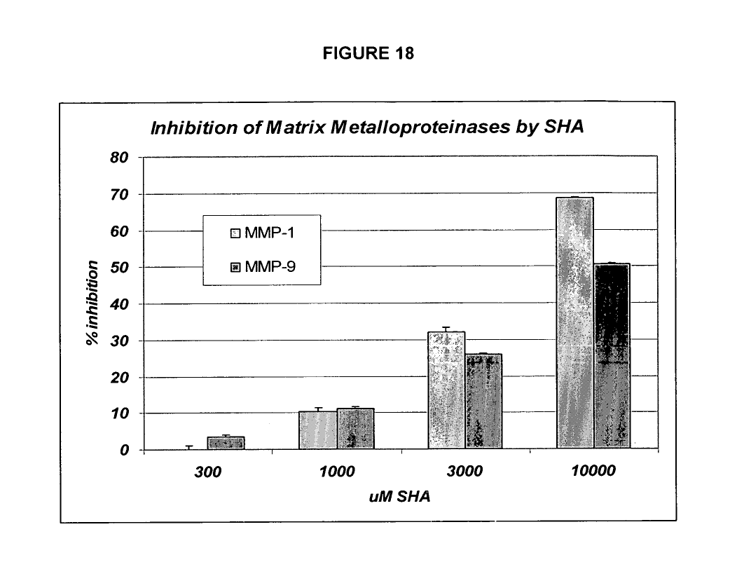

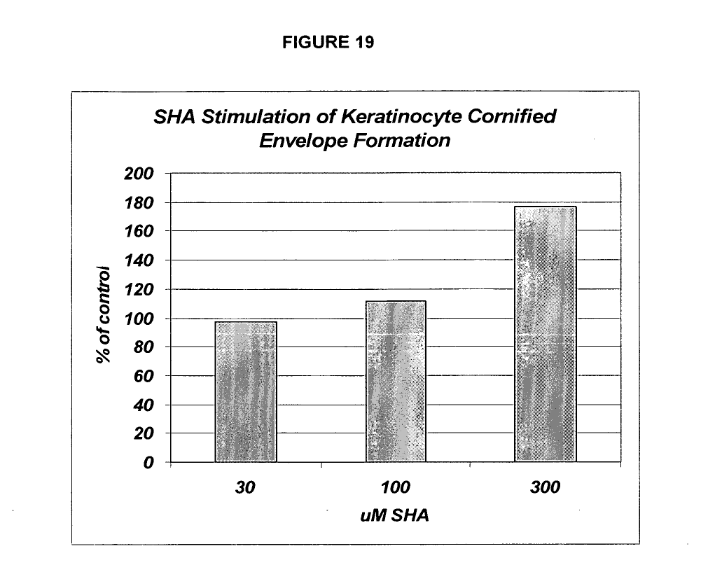

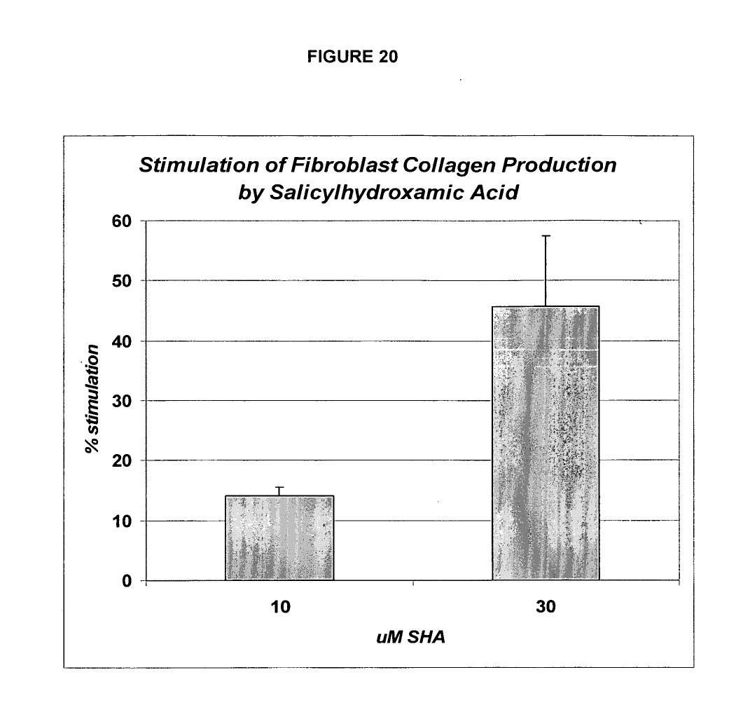

The skin lightening effect of SHA was determined by measuring the inhibition of tyrosinase since tyrosinase is the enzyme that catalyzes the rate limiting steps in melanin synthesis. Inhibition of mushroom tyrosinase was assayed by measuring conversion of tyrosine to DOPAchrome (OD475) in the presence of varying concentrations of test sample. FIG. 15 is a bar graph that displays the inhibition of tyrosinase by SHA. Thirty nanomolar SHA inhibited over 30% of the activity. The IC₅₀ is 60 nM.

Liposomes as a Delivery Vehicle

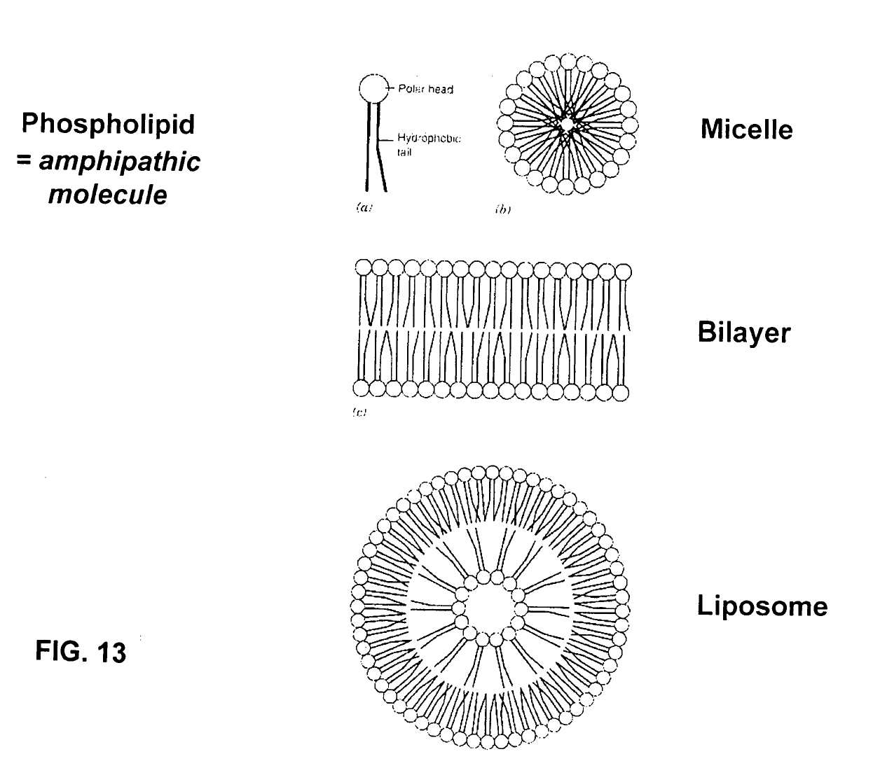

According to one embodiment of the present invention, liposomes are used as a delivery agent for the antioxidant-hydroxamate combination. Liposomes are microscopic spherical vesicles that form when phospholipids are hydrated. As shown in top left figure of FIG. 13, a phospholipid has a polar head connected to a hydrophobic tail. Phospholipids arrange themselves in micelles, with the polar heads directed outwards and the hydrophobic tails coming together in the center of the structure, as indictaed in the top right figure of the drawing. Phospholipids can also form bilayer sheets, as indicated in the middle figure of FIG. 13, where the molecules align side by side in like orientation, with the heads forming the surfaces of the sheet and the tails directed inwards. These sheets are joined tails-to-tails to form a bilayer membrane, which encloses some of the water in a phospholipid sphere.

Normal liposomes form a multilamellar structure of concentric phospholipid spheres separated by layers of water, as shown in the bottom figure of FIG. 13. Because of this, the inside useful volume that can accommodate water-soluble ingredients is low. Collaborative liposomes, commercially available from Engelhard Corporation, utilizing an ultra high-shear processing, form a unilamellar structure, a single phospholipid bilayer sphere enclosing water.

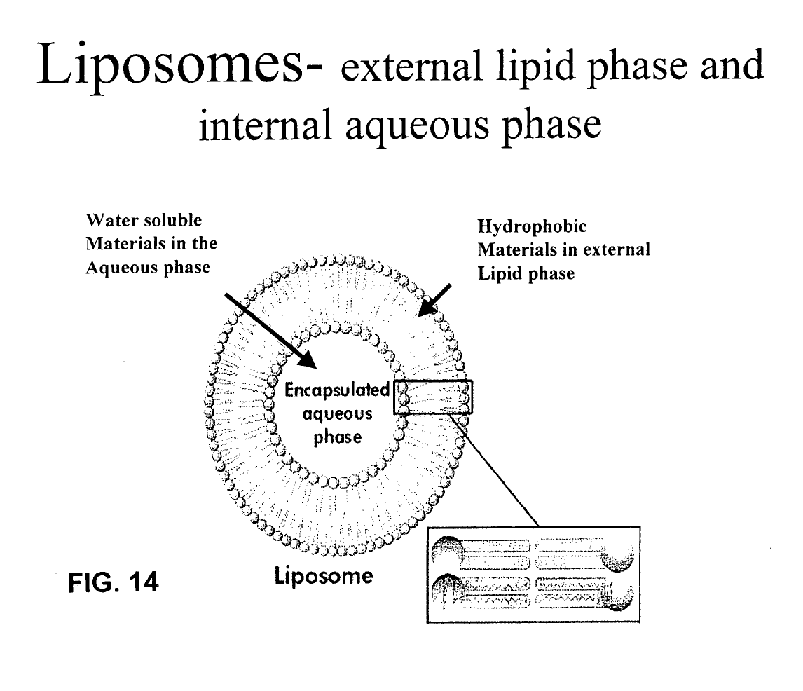

A phospholipid bilayer sphere according to an embodiment of the invention is depicted in FIG. 14. The bilayer sphere comprises a bilayer sheet that has folded into a sphere, with the polar heads comprising an outer surface and an inner surface surrounding an encapsulated aqueous phase, where water soluble substances can be stored. The tails are aligned in between the surfaces, forming an external lipid phase, as indicated in the box at the lower right, and hydrophobic materials can be stored in this external lipid phase.

The collaborative liposomes of the present invention may be prepared by the following process steps, which are carried out at room temperature (25° C.) under 1 atmosphere pressure of argon gas (to limit any opportunity for oxidation):

(a) Use a marine or propeller-type mixer to disperse the membrane-forming lipid in the total quantity of water available to the formulation. According to an embodiment of the invention, the mixing occurs at 500 to 1500 rpm and lasts for 1 hour. The remaining water-insoluble phases are then added to the wetted lipids, along with the salicylhydroxamate and any germicides or preservatives. Any additional cosmetic excipients or active ingredients are then added to the batch. According to another embodiment of the invention, the mixing can continue at 500 to 1500 rpm for 1 hour with caution to avoid vortex and entrainment of air.

(b) In order to facilitate hydration of the batch prior to high-shear processing, homogenize the batch with a rotor/stator-type homogenizer running at 3,000 rpm for 30 minutes.

(c) At this stage the batch is subjected to high-shear processing, specifically microfluidization. In this process, the liquid is extruded through two 100 micron pores. The jetting streams collide within an interaction chamber, and then the liquid is extruded to ambient pressure. The combination of shear and pressure change completes the dispersion of the insoluble phases and produces liposomes as verified by freeze-fracture electron microscopy.

(d) Finally, any necessary additional cosmetic ingredients meant to occupy solution volumes outside the liposomes are added. Such materials might include thickeners or other rheologic agents, emollients, humectants and germicides.

The above procedure is generic to all of the specific types of compositions, and may be modified as necessary to achieve the preparation of a specific final product. Different ingredients may require modification in the order of addition of materials, depending on whether the intention is to locate the ingredient internal or external to the liposome, whether the ingredient is water-soluble, or whether the ingredient behaves as an acid or base.

Besides being much smaller than multilamellar liposomes, these unilamellar liposomes are of uniform size, usually 200 nanometers or less in diameter. Additionally, the ultra high-shear mixing conditions allow the phospholipids to align and orient themselves into bilayers with more regularity than is possible with other processing techniques, making them much more stable.

Water-soluble materials dissolved in water in which the phospholipids are hydrated will be trapped in the aqueous center of the liposome, while fat-soluble materials, such as oils, will adhere to the liposome wall, which is a phospholipid membrane. Thus, the water soluble hydroxamate derivatives will be trapped in the aqueous layer of the liposome, while the oil soluble antioxidants, such as vitamin E, coenzyme Q10, NDGA etc., will be in the liposomal phopsholipid membrane.

This arrangement of the hydroxamates and the anti-oxidants will not only keep these material stable, but apart from each other before the liposome comes in contact with the skin, enabling a controlled delivery of the active ingredients. Liposomes can deliver actives specifically to cellular sites, allowing lower levels of active ingredients than conventional formulations. In addition, the phospholipid membranes merge with cellular membranes, releasing actives over extended periods of time. Phospholipids used for liposomes are biocompatible, biodegradable, and non-toxic. Thus, these phospholipids do not adversely affect skin physiology or biochemistry. The released hydroxamates and the antioxidants will be available readily to the skin. This ensures that the longer term activities of the hydroxamate and antioxidants will be available for skin functions.

Liposomes can make soluble recalcitrant compounds and can deliver a wide range of active ingredients. They are versatile, can encapsulate wide range of active ingredients, including water-soluble and fat-soluble molecules, different molecular sizes, and different classes of molecules, such as lipids, proteins, carbohydrates, nucleic acids, etc. Bioactive compounds, such as vitamin A and vitamin E, are protected from exposure to atmospheric oxygen, thereby stabilizing them. Antioxidants, including water soluble antioxidants such as vitamin C, polyphenols, etc., are especially unstable molecules. The stability of these molecules is increased by incorporating them into liposomes. By controlling the hydration and limiting contact with other components in the formulation, such as surfactants, liposomes can stabilize proteins and enzymes. Many lipid soluble antioxidants that are unstable in aqueous phase are stabilized in a liposomal preparation.

The liposome wall is very similar, physiologically, to the material of cell membranes. When cosmetic containing liposomes are applied to the skin, the liposomes are deposited on the skin, merge with the cellular membranes and release their payload of active materials into the cells over a long period of time, providing a controlled release of the payload. By controlling the delivery, the concentration of active contact with skin can be minimized for toxic or high effective molecules, such as retinoids that are effective at low nanomolar concentrations. Slow delivery is important for providing longer-lasting benefits to the skin from the hydroxamate derivatives and antioxidants.

By changing the physical and chemical characteristics of the liposome, such as chain length, saturation of the phospholipid, incorporating other lipids such as glycolipids and sterols, etc., or by changing the pH or temperature, one can change the kinetics of the payload release. The advantage of using liposomes is that one can incorporate a water soluble and an oil soluble hydroxamate derivative and antioxidant within the same product. For example, vitamin E and vitamin C can be incorporated into same product without each other affecting the others stability. The vitamin E will be incorporated within the phospholipid layer and the vitamin C will be within the water phase in the liposome. Upon application to skin, these two antioxidants will be released simultaneously on the surface of skin, thereby providing synergistic benefits.

According to another embodiment of the invention, a cationic liposome vehicle as disclosed in U.S. Pat. No. 5,874,105, can deliver these actives to hair follicles and sebaceous glands for benefits in hair growth, sebum suppression, and prevention of acne formation. These cationic liposomes are available under the trade name CATEZOMES®, a registered trademark of Engelhard Corporation.

### EXAMPLES

The following examples illustrate skin care compositions according to the present invention. The compositions can be processed in conventional manner, and are suitable for cosmetic use. In particular, the compositions are suitable for application to wrinkled, lined, rough, dry, flaky, aged and/or UV-damaged skin to improve the appearance and the feel thereof as well as for application to healthy skin to prevent or retard deterioration thereof.

1. This example illustrates a high internal phase water-in-oil emulsion incorporating a composition according to an embodiment of the present invention.

| | % w/w |
|---|---|
| Salicylhydroxamate | 0.1 |
| NDGA | 0.001 |
| Green tea polyphenols | 0.1 |
| 1,3-dimethyl-2-imidazolidinone | 0.2 |
| Brij ® 92* | 5 |
| Bentone 38 | 0.5 |
| MgSO₄ 7H₂ O | 0.3 |
| Butylated hydroxy toluene | 0.01 |
| Perfume | qs |
| Water | to 100 |

*Brij 92 is polyoxyethylene (2) oleyl ether and is a registered trademark of ICI Americas.

2. This example illustrates an oil-in-water cream according to an embodiment of the present invention.

| | % w/w |
|---|---|
| Liposome containing salicylhydroxamate/NDGA | 2 |
| Mineral oil | 4 |
| 1,3-dimethyl-2-imidazolidinone | 1 |
| Alfol ® 16RD* | 4 |
| Triethanolamine | 0.75 |
| Butane-1,3-diol | 3 |
| Xanthan gum | 0.3 |
| Perfume | qs |
| Butylated hydroxy toluene | 0.01 |
| Water | to 100 |

*Alfol 16RD is cetyl alcohol and is a registered trademark of Condea Vista Co.

3. This example illustrates an alcoholic lotion incorporating the composition according to the invention.

| | % w/w |
|---|---|
| Peptide hydroxamate | 0.1 |
| Antioxidant (vitamin C) | 2 |
| Plant isoflavonoid | 0.5 |
| 1,3-dimethyl-2-imidazolidinone | 0.1 |
| Ethanol | 40 |
| Perfume | qs |
| Butylated hydroxy toluene | 0.01 |
| Water | to 100 |

4. This example illustrates another alcoholic lotion containing the inventive composition.

| | % w/w |
|---|---|
| Salicylhydroxamate | 0.5 |
| Vitamin E | 0.1 |
| Green tea polyphenol | 0.5 |
| 1,3-dimethyl-2-imidazolidinone | 0.01 |
| Ethanol | 40 |
| Antioxidant | 0.1 |
| Perfume | qs |
| Water | to 100 |

5. This example illustrates a suncare cream incorporating the composition of the invention:

| | % w/w |
|---|---|
| Liposome containing 0.5% of a | 2 |
| hydroxamate and 0.1% of an antioxidant | |
| 1,3-dimethyl-2-imidazolidinone | 0.2 |
| Silicone oil 200 cts | 7.5 |
| Glycerylmonostearate | 3 |
| Cetosteryl alcohol | 1.6 |
| Polyoxyethylene-(20)-cetyl-alcohol | 1.4 |
| Xanthan gum | 0.5 |
| Parsol 1789 | 1.5 |
| Octyl methoxycinnate (PARSOL MCX) | 7 |
| Perfume | qs |
| Color | qs |
| Water | to 100 |

6. This example illustrates a non-aqueous skin care composition incorporating the inventive combination.

| | % w/w |
|---|---|
| Antioxidant mixture of vitamin | 5 |
| C palmitate, vitamin E, | |
| A mixture of salicylhydroxamate | 1 |
| and trypotphan hydroxamate | |
| 1,3-dimethyl-2-imidazolidinone | 1 |
| Silicone gum SE-30 | 10 |
| Silicone fluid 345 | 20 |
| Silicone fluid 344 | 50.26 |
| Squalene | 10 |
| Linoleic acid | 0.01 |
| Cholesterol | 0.03 |
| 2-hydroxy-n-octanoic acid | 0.7 |
| Herbal oil | 0.5 |
| Ethanol | 2 |

It should be understood that the specific embodiments of the invention herein illustrated and described are intended to be representative only, as the invention may be modified and practiced in different but equivalent manners apparent to those skilled in the art having the benefit of the teachings herein. Furthermore, no limitations are intended to the details of composition herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. A cosmetic composition comprising:
one or more hydroxamate derivatives in an amount of about 0.001% to about 10.0% by weight; and
one or more antioxidants in an amount of about 0.00001% to about 10% by weight.

2. The cosmetic composition of claim 1, further comprising a liposomal vehicle encapsulating said hydroxamate derivatives and said antioxidants.

3. The composition of claim 2, wherein the liposomal vehicle comprises a cationic liposome.

4. The composition of claim 2, wherein the hydroxamate derivatives are contained within in the aqueous layer of the liposomal vehicle, while the antioxidants are contained in the liposomal vehicle membrane.

5. The composition of claim 1, wherein the hydroxamate derivatives include metal ion complexes.

6. The composition of claim 5, wherein the metal ions are selected from the group comprising of Zn, Cu, Mn, Mg, Sr, B, Al, Rb and Ca.

7. The composition of claim 1, wherein the hydroxamate derivatives comprise hydroxamates of salicylic acid and its derivatives, hydrobenzioc acid, tryptophan, amino acids, peptides containing from 1 to 6 amino acids, peptide mimetics, alpha hydroxy acids, dicarboxylic acids, substrate-analogue peptides of matrix metallo proteases containing aminomalonic acid, and monohydroxamates of aspartic and glutamic acids.

8. The composition of claim 1, wherein the antioxidants include vitamin C and its derivatives, vitamin E and its esters, ubiquinone, coenzyme Q10, lipoic acid, and polyphenolic compounds.

9. The composition of claim 8, wherein the polyphenolic antioxidants include caffeic acid, ferulic acid, quercetin, apigenin, genistein, resveratrol, nordihydroguaiaretic acid, camosic acid, ursolic acid, rosemarinic acid, silymarin, epicatechin, epicatechin-3-gallate, epigallocatechin, epigallocatechin gallate, procyanidins, proanthocyanidins, gallotannins, ellagotannins, and pycnogenol.

10. The composition of claim 1, wherein the antioxidant is nordihydroguaiaretic acid, and the hydroxamate derivative is salicylhydroxamate.

11. The composition of claim 1, wherein the antioxidant is present in an amount of about 0.001% to about 5% by weight, and the hydroxamate is present in an amount of about 0.01% to about 1% by weight.

12. The composition of claim 11, wherein the antioxidant is present in an amount of about 0.01% to 5% by weight, and the hydroxamate derivative is present in an amount of about 0.01% to 0.5% by weight.

13. A method of boosting collagen and promoting anti-aging in skin comprising the step of using a combination of a hydroxamate derivative and antioxidant on the skin in a cream or lotion or a wash off type product formulation.

14. The method of claim 13, wherein the antioxidant is nordihydroguaiaretic acid, and the hydroxamate derivative is salicylhydroxamate.

15. A method of providing anti-inflammatory and anti oxidant benefit to skin comprising the step of using a combination of a hydroxamate derivative and antioxidant on the skin in one of a cream, lotion, and a wash off type product formulation.

16. The method of claim 15, wherein the antioxidant is nordihydroguaiaretic acid, and the hydroxamate derivative is salicylhydroxamate.

17. A method of preventing acne formation and suppression of sebum secretion in skin comprising the step of using a combination of a hydroxamate derivative and antioxidant on the skin in one of a cream, lotion, and a wash off type product formulation.

18. The method of claim 17, wherein the antioxidant is nordihydroguaiaretic acid, and the hydroxamate derivative is salicylhydroxamate.

19. A method of brightening skin and inhibiting tyrosinase in skin comprising the step of using a combination of a hydroxamate derivative and antioxidant on the skin in one of a cream, lotion, and a wash off type product formulation.

20. The method of claim 19, wherein the antioxidant is nordihydroguaiaretic acid, and the hydroxamate derivative is salicylhydroxamate.

21. A cosmetic composition comprising:
about 0.1% to about 10% by weight of a membrane-forming lipid phase;
about 0.00001% to about 10% by weight of nordihydroguaiaretic acid; and
about 0.001% to about 10% by weight of salicylhydroxamate; and
about 40% to about 99.8% by weight of water, wherein the composition is subjected to high sheer and high pressure processing to form a liposome from the membrane-forming lipid phase.

22. The composition of claim 21, wherein the nordihydroguaiaretic acid is present in an amount of about 0.001% to about 5% by weight, and the salicylhydroxamate is present in an amount of about 0.01% to about 1% by weight.

23. The composition of claim 22, wherein the nordihydroguaiaretic acid is present in an amount of about 0.01% to about 5% by weight, and the salicylhydroxamate is present in an amount of about 0.01% to about 0.5% by weight.

24. The composition of claim 21, wherein the salicylhydroxamate includes metal ion complexes formed from ions selected from the group comprising of Zn, Cu, Mn, Mg, Sr, B, Al, Rb and Ca.

25. The composition of claim 21, wherein the liposome is a cationic liposome.
